(19) United States
(12) Patent Application Publication
(10) Pub. No.: US 2005/0112623 A1
(43) Pub. Date: May 26, 2005

(54) Bio-cell chip

(76) Inventors: Dong Soon Lee, Seoul (KR); Jee Hyung Lee, Seoul (KR)

Correspondence Address: HESLIN ROTHENBERG FARLEY & MESITI PC, 5 COLUMBIA CIRCLE, ALBANY, NY, 12203, US

(21) Appl. No.: 10/920,054

(22) Filed: Aug. 17, 2004

Related U.S. Application Data
(63) Continuation of application No. PCT/KR03/00322, filed on Feb. 14, 2003.

(30) Foreign Application Priority Data
Feb. 18, 2002 (KR) 10-2002-0008394

(51) Int. Cl. C12Q 1/70; C12Q 1/68; C12M 1/34

(52) U.S. Cl. 435/6; 435/287.2

(57) ABSTRACT

The present invention relates to bio-cell chips and methods for making the same.

### TECHNICAL FIELD

The present invention relates to bio-cell chips and methods for preparing the same.

### BACKGROUND ART

Recently, an enormous amount of detailed gene information has been published, so that the needs for large-scale genomic screening methods have been increasing. As well-known examples, high-density cDNA arrays or DNA chips made it possible to detect thousands of genes simultaneously. With the development of these powerful methods, using knowledge from genomic discovery in clinical area such as clinical diagnosis or prognosis assessment, the development of personalized medicine for patient individual and preventive therapy for the ones with disease-associated genes has become urgent necessity. Even among cancer cells with same histologic features, genetic alterations involved in inducing or progressing the cancer cells are different and each case of genetic alterations results in various disease prognoses and different responses to anticancer drugs. Thus, in order to predict such clinical responses, it becomes essential to detect simultaneously plenty of genes of cancer cell or tissue primarily obtained from a patient.

At present, in all biotechnological research, DNA, RNA and protein have been detected in an isolated form from a cell after the cell was destroyed, and thus positional and biological significance of these bio-samples in the cell has been ignored. If it is possible to detect the changes of DNA, RNA and protein remained in the cell without cell destruction, the results will have biological significance. However, there exist some limitations for conducting research such as it is not easy to preserve cells in intact forms without damage and a great number of cells are needed for various kinds of assays.

When a sample obtained from a patient, for example, blood, tumor specimen or cytology specimen, is used for gene detection, the amount of specimen could be a restricting factor because it is too small to perform various assays. Therefore, if a cell array only with the minimal number of cells required for the test can be produced, the shortage problem of sample amount can be solved and numerous genes can be detected at the same time with low-cost.

### SUMMARY OF THE INVENTION

The present invention provides bio-cell chips in which cells are arrayed, and methods for preparing the same by spot-spraying cells into a small space on the biochip substrate. The bio-cell chips according to the present invention are suitable for performing hundreds of assays with a small amount of sample and subjecting hundreds of experimental objects to the same condition while conducting assays.

The object of the present invention is that thousands of cells can be detected for gene or protein expression at the same time.

### DISCLOSURE OF THE INVENTION

The present invention relates to bio-cell chips and methods for preparing the same.

The present invention provides bio-cell chips in which cells are arrayed and fixed, replacing DNA in conventional DNA chips. That is to say, the biochips in which plenty of cells are arrayed and fixed in a small space are provided. The bio-cell chips according to the present invention may have between 100 and 2000 cells, preferably between 100 and 500 cells arrayed and fixed in a small space, for example, 70 mm×30 mm.

A supporting material, that is, a chip substrate, on which the bio-cell chips of the present invention can be constructed by arraying and fixing cells may be those employed in conventional biochips. Examples include plastics, silicone and glass slides with low background fluorescence.

The bio-cell chips of the present invention may be produced according to the established production methods for the conventional biochips such as DNA chips etc. One major difference from the conventional production methods using DNA, RNA or protein is that in the case of cell, to array and fix a large number of cells in a small space, cell suspension stored in fixing agents has not to be spread out. Therefore, special measures have to be done.

The above problem has been solved by the present invention that provides separate rooms for each cell to be fixed apart from each other. The method forming the separate rooms is:
a) to build septum between samples,
b) to produce wells allowing samples to be dispensed into, on the surface of a supporting material, that is, the surface of a chip substrate constituting a chip by cutting it in a uniform shape, or
c) to produce grooves on the surface of a supporting material, that is, the surface of a chip substrate constituting a chip by engraving it.

The difference between a well and a groove lies on that, because the depth of well cannot be increased to a desirable amount, a groove is made by cutting the width of hole to create a prolonged hollow. Thus, it is desirable to produce grooves when a relatively large number of cells per a sample are to be applied. The materials for building septum includes, for example, cement such as rubber cement, and sticker. These materials should be completely removed with ease after the fabrication of the bio-cell chip and have no influence on in situ hybridization staining or shape of the cells.

In addition to the above methods of forming the rooms, to prevent the solution with cell suspension on the surface of the supporting material, that is, the surface of the chip substrate constituting the chip from spreading out, an alternative method that comprises providing special treatments to modify surface characteristics and then spraying a sample, can be employed. Above special treatments include, for example, treating the surface with a sticky material such as polymers.

Thus, in the bio-cell chips according to the present invention, cells can be
a) located and fixed in separate rooms or
b) arrayed by spotting or spraying the cell suspension, and fixed separately by aid of a sticky material treated on the surface of a chip substrate.

More specifically, the above rooms can be formed by septum, wells or grooves engraved on the surface of the chip substrate, and the above septum can be made up with cement or sticker.

In the production of the bio-cell chips according to the present invention, methods of spraying samples of cells include operating an automated robot system that allows at least 500 or an average of 2000 cells to be loaded at the same time. The above robot system can be operated two different ways. One is to load different kinds of samples of at least 500 or an average of 2000 on a slide and to apply one kind of an in situ hybridization agent. The other is to react different kinds of in situ hybridization reaction reagents with one kind of samples using the smallest amount automatic system.

Each sample has its own number and operating a program associated with a reading microscope makes it possible to find and approach target samples automatically. Determinations of results can be carried out on the whole samples throughout a slide automatically using a program inputted with detection data.

Thus, the present invention do not cause slide to slide variation that is a major concern in in situ hybridization technique. In practical assay or clinical research, the conditions subjected to each slide may be not identical, which often makes results assessment difficult. Standardization can be achieved by using bio-cell chips applied almost about 100 to 5000 samples on a slide. The bio-cell chips according to the present invention provide simultaneous detection of gene expression for thousands of cells.

The bio-cell chips of the present invention have made it possible to detect gene expression for a great number of samples with an extremely small amount of sample and a small amount of reagent at the same time, which has brought innovations in terms of cost, time and effort in the field of a disease diagnosis and drug effect evaluation for cancer or genetic disease as well as has greatly improved reliability of the results.

Following applications are also available using the bio-cell chips according to the present invention:
a) The bio-cell chips will be used for a mass screening in the diagnosis of tumor of which cells can be easily obtainable from a human body with non-invasive methods. For example, in case of lung cancer or bladder cancer, cells collected from sputum or urine can be stained, and then cancer cells with specific genetic alterations or with specific antigens can be detected using an automated analysis system with high accuracy.
b) Tumor cell lines array chips in which various tumor cell lines will be laid on one place can be fabricated, and standardization of the assays will also be achieved.
c) Bio-cell chips containing tumor cells collected and isolated from a patient's tumor can be fabricated and also provided to basic biological researchers.
d) Standardization of a single cell-PCR or in situ hybridization (ISH) can be achieved.
e) Minimal residual leukemic cells can be detected.
f) The development of systems to predict anticancer drug susceptibility can be accomplished.
g) The bio-cell chips will be useful for the assessment of drug susceptibility.

### BEST MODE FOR CARRYING OUT THE INVENTION

While the present invention will be illustrated in more detail by the ways of a following example and an experimental example, it will be understood that it is not the intent to limit the invention to these examples.

### EXAMPLE 1

100 samples were applied on a slide manually or using a multi-dispenser offering the ability to dispense a small quantity of cell suspension, and then molecular cytogenetic study and detection of genetic changes were conducted. Staining was successfully completed with a small amount of sample and a small amount of reagent.

100 septa were made on a glass slide with rubber cement, and then were dried for 10 min. 1 μl of cell suspension was dispensed into each well. When the dispensed sample was dried out over 20 min, rubber cement was completely removed and in situ hybridization staining was performed.

The amount of the reagent typically used was 10 μl per one slide. Detections of genetic changes of 100 samples were conducted with the same amount of the reagent required in one sample.

### EXPERIMENTAL EXAMPLE 1

Bio-cell chips are also available for cancer diagnosis using cells obtained from a large number of patients. For lung cancer diagnosis or follow-up study, cells of sputum are examined. While usually PAP staining is used and the cells are examined one by one, it is often difficult to discriminate between a cancer cell and a dysplastic cell morphologically. If it is possible to detect specific genetic alterations in lung cancer cells of the Korean people, specificity of diagnosis can be increased. A bio-cell chip containing each cell isolated from patients' sputum is fabricated and then specific genetic alterations are detected using in situ hybridization technique. It is possible to diagnose lung cancer for a massive group of patients, while also not requiring manual labor. Cells obtainable from a human body without any medical procedure include bladder cancer cells naturally excreted with urine. A bio-cell chip was fabricated with cells isolated from urine samples and then specific genetic alterations of bladder cancer are detected using in situ hybridization technique. It is possible to accomplish massive diagnosis and follow-up study for bladder cancer without calling on patients to the hospital for study, while also not requiring a special procedure.

Following published references are the inventors' research papers presenting examples in which various assays have been performed with the bio-cell chips according to the present invention:
(1) Kyoung Un Park, Cha Ja She, Hee Young Shin, Hyo Seop Ahn, Chong Jai Kim, Byung Kyu Cho, Han Ik Cho, Dong soon Lee. The Low Incidences of TEL/AML1 fusion and TEL Deletion in Korean hildhood Acute Leukemia by Extra-signal Fluorescence In Situ Hybridization. Cancer Genetics and Cytogenetics The Low Incidences of TEL/AML1 fusion and TEL Deletion in Korean hildhood Acute Leukemia by Extra-signal Fluorescence In Situ Hybridization. Cancer Genetics and Cytogenetics, 2001, 126;73-77.
(2) Dong Soon Lee, Sunny Kim, Eul Ju Seo, Chan Jung Park, Hyun Sook Chi, Byoung Hak Yoon, Wo Ho Kim, Han Ik Cho. Predominance of trisomy 1 q in myelodysplastic syndromes in Korea; Is it a ethnic difference?—3 year-experience of multi-center study—Genetics and Cytogenetics, 2001, 129;73-77.
(3) Dong Soon Lee, Eu Chong Kim, Byoung Hak Yoon, Eun Kyung Ko, Sun Yang Park, Woo Hoo Kim, Jong Hyun Yoon, Han Ik Cho. Can Minor bcr/abl Translocation in Acute Leukemia be Discriminated from Major bcr/abl by Extra-Signal FISH Analysis Haematologica 2001 86;991-992.
(4) Kyoung Un Park, Dong Soon Lee, Hye Seung Lee, Chong Jai Kim, and Han Ik Cho. Granulocytic Sarcoma in MLL-positive Infant Acute Myelogenous Leukemia: Fluorescence In Situ Hybridization Study of Childhood Acute Myelogenous Leukemia for Detecting MLL Rearrangement. American Journal of Pathology. 2001:159;2001-2016.

1. A bio-cell chip of which cells are arrayed and fixed on the substrate of the chip.

2. The bio-cell chip according to claim 1, wherein each cells is located and fixed in separate rooms.

3. The bio-cell chip according to claim 2, wherein the rooms are formed by septum, wells or grooves engraved on the surface of a chip substrate.

4. The bio-cell chip according to claim 3, wherein the septum is made up with cement or sticker.

5. The bio-cell chip according to claim 4, wherein the cement is rubber cement.

6. The bio-cell chip according to claim 1, wherein cells are arrayed and fixed separately by aid of a sticky material treated on the surface of a chip substrate.

7. A method of making a bio-cell chip of claim 1, comprising loading each cell and then applying one kind of an in situ hybridization reagent using an automated robot system.

8. A method of making a bio-cell chip of claim 1, comprising loading each cell and then reacting different kinds of in situ hybridization reagents with target samples of the cells using the smallest amount automated system and also using an automated robot system.
